SEQUENCE LISTING (2 sequences)

SEQ ID NO: 1 - protein, 341 aa, Bacteriophage A511
MVKYTVENKIIAGLPKGKLKGANFVIAHETANSKSTIDNEVSYMTRNWKNAFVTHFVGGGGRVVQVANVNYVSWGAGQYANSYSYAQVELCRTSNATTFKKDYEVYCQLLVDLAKKAGIPITLDSGSKTSDKGIKSHKWVADKLGGTTHQDPYAYLSSWGISKAQFASDLAKVSGGGNTGTAPAKPSTPAPKPSTPSTNLDKLGLVDYMNAKKMDSSYSNRDKLAKQYGIANYSGTASQNTTLLSKIKGGAPKPSTPAPKPSTSTAKKIYFPPNKGNWSVYPTNKAPVKANAIGAINPTKFGGLTYTIQKDRGNGVYEIQTDQFGRVQVYGAPSTGAVIKK

SEQ ID NO: 2 - DNA, 1023 bp, Bacteriophage A511
atggtaaaatataccgtagagaacaaaattattgcaggattacctaaaggtaaactaaaaggggctaactttgttattgctcatgaaactgcaaatagcaagtctactattgacaatgaagtaagctacatgactaggaactggaagaacgcatttgtaactcactttgtaggtggcggaggtagagtcgttcaggttgctaatgtaaactatgtttcttggggagcaggtcagtatgctaactcttattcctatgcgcaggtagagttgtgccgtacaagtaatgcaactacatttaagaaagactatgaagtgtactgtcaattactagtagacctagctaaaaaagcaggtatccctattacacttgactctggtagtaaaactagtgataaaggtattaaatcccataaatgggttgctgataagctaggaggaacaacacaccaagacccatatgcttacttaagctcatggggtattagtaaagcacaatttgctagtgacttggctaaagtatctggcggaggaaacacaggaacagcgccagctaaaccaagcacaccagcacctaaaccaagcacaccatctactaacctagacaaacttggcttagtagactacatgaacgctaagaaaatggactctagctacagtaacagagataagttagctaaacagtatggtattgctaactattcaggaacagctagccagaacactacactccttagtaaaattaaaggaggagcacctaaaccaagcacaccagcacctaaacctagtacatctacagctaagaaaatttatttcccaccaaataaaggaaactggtctgtgtatccaacaaataaagcacccgttaaggctaatgctattggtgctattaaccctactaaattcggaggattgacttacactatccaaaaagatagaggaaacggtgtatacgaaatccaaacagaccaattcggcagagttcaagtctatggtgcacctagtacaggagcagttatcaaaaaa

(19) United States
(12) Patent Application Publication
(10) Pub. No.: US 2011/0243916 A1
(43) Pub. Date: Oct. 6, 2011

(54) MODIFIED ENDOLYSIN PLY511

(76) Inventors: Anna Scherzinger, Bernried (DE); Martina Beissinger, Regensburg (DE); Holger Grallert, Weilheim (DE)

(21) Appl. No.: 12/992,224

(22) PCT Filed: May 14, 2009
(86) PCT No.: PCT/EP09/55869

§ 371 Date: Jun. 21, 2011

(30) Foreign Application Priority Data
May 14, 2008 (DE) 10 2008 023 447.8
Aug. 19, 2008 (DE) 10 2008 038 370.8

(51) Int. Cl. A61K 38/48; C07H 21/00; C12N 9/50; C12N 15/63; C12N 1/00; A23L 3/3571; C12Q 1/37; A61P 31/04

(52) U.S. Cl. 424/94.63; 536/23.2; 435/219; 435/320.1; 435/243; 426/61; 435/23; 426/321

(57) ABSTRACT

The present invention relates to polypeptides with a changed amino acid sequence on at least one amino acid position compared to the amino acid sequence according to SEQ ID NO: 1. The present invention further relates to the nucleotide sequences encoding the polypeptide, vectors, comprising the nucleotide sequences and host cells for expression of the polypeptide. The present invention further relates to the use of the polypeptides as a human, veterinary medical or diagnostic substance, in food, in cosmetics, as disinfectant or in the environmental field.

The present invention relates to polypeptides with a changed amino acid sequence on at least one amino acid position compared to the amino acid sequence according to SEQ ID NO: 1. The present invention further relates to the nucleotide sequences encoding the polypeptide, vectors, comprising the nucleotide sequences and host cells for expression of the polypeptide. The present invention further relates to the use of the polypeptides as a human, veterinary medical or diagnostic substance, in food, in cosmetics, as disinfectant or in the environmental field.

Listeria are widely spread human and animal pathogen bacteria in the field of food, causing the disease Listeriosis. Frequently food such as fish, meat and milk products is contaminated with Listeria. The class Listeria comprises six different species with 16 different serotypes. In detail, these are L. monocytogenes with the serotypes 1/2a, 1/2b, 1/2c, 3a, 3b, 3c, 4a, 4ab, 4b, 4c, 4d, 4e, 7; L. innocua with the serotypes 3, 6a, 6b, 4ab, U/S; L. ivanovii with the serotype 5; L. seeligeri with the serotype 1/2a, 1/2b, 1/2c, 4b, 4c, 4d, 6b; L. welshimeri with the serotypes 1/2a, 4c, 6a, 6b, U/S and L. grayi with the serotype Grayi. Both species L. monocytogenes and L. ivanovii are known to be pathogen. A third species, L. seeligeri, is considered to be non-pathogenic; however, one case is known where L. seeligeri caused Meningitis in a human being. The remaining species are considered to be nonpathogenic. Approximately 90% of Listeriosis is attributed to L. monocytogenes Serotype 1/2a, 1/2b and 4b (Wing E J & Gregory S H, 2002, Listeria monocytogenes: Clinical and Experimental Update, J Infect Diseases 185 (Suppl 1): S18-S24).

Although Listeriosis is a rare disease, it has to be taken serious because of the severity of the disease and the high rate of mortality. Although only a small portion of the food related diseases is caused by Listeria (approx. 1% in USA), almost 30% of the annually fatal diseases, caused by food pathogens, are caused by this germ. Affected are mainly immune suppressed persons, e.g. older people, diabetes patients, cancer patients and/or aids patients. Pregnant women and the yet unborn child represent approx. 25% of all cases of Listeriosis patients. Due to their ability to pass the blood-brain barrier or the placenta barrier, Listeria could cause meningitis, encephalitis, abortion and still birth (Wing E J & Gregory S H, 2002, Listeria monocytogenes: Clinical and Experimental Update, J Infect Diseases 185 (Suppl 1): S18-S24; Doyle M E, 2001, Virulence Characteristics of Listeria monocytogenes, Food Research Institute, October 2001).

Listeria is well-adapted for the survival in the environment of food production. They are tolerant towards weak acids and they are capable to reproduce at relatively high salt concentrations and at temperatures from 1° C. to 45° C. The main source of infection is food, especially if it is not heat treated before consumption, e.g. many milk products, smoked fish, meat products and in an increasing degree ready-to-eat products (especially products containing meat). The contamination with Listeria frequently takes place in food processing (removal from the cooking containers, cutting, garnishing, packing, etc.). Food produced with the help of starter cultures not treated with heat (e.g. raw milk cheese, salami), could also be contaminated by the starter culture or the raw material itself or also during maturation or storage. Whereas in the USA there is zero tolerance for L. monocytogenes in ready-to-eat food, many European countries or Canada allow contamination of certain food with Listeria of up to 100 CFU (Colony forming unit)/g food. However, in any case the food has to be analyzed for contamination of Listeria. A lot of food, e.g. seafood, smoked salmon, milk products or also ready-to-eat raw products only have a short shelf life. Therefore, cost intensive product recalls frequently occur, if a contamination with Listeria or a contamination above the allowed limit is detected in these products after delivery.

For this reason there is high interest to provide methods for the detection as well as decontamination of Listeria. Furthermore, the application of antimicrobic substances is important, to inhibit the growth of Listeria as well as to kill present Listeria. EP 0781349 describes amongst others the Listeria Phage lysin from the phage A511, Ply511, which could be used for the above mentioned application. Due to its wide host spectrum Ply511 could be used against a multitude of Listeria serotypes, but they are inappropriate for the use in food due to the relatively low stability. Turner et al. (2007, Syst. And Appl. Microbiol., 30, 58-67) refer to proteolysis problems in the expression of Ply511 in Lactobacilli potentially applied in food and propose that the stability of Ply511 should be increased for the respective application. However, no indication for solutions is disclosed by Turner concerning an increase of the Ply511 stability.

Thus, the object of the present invention is to provide more stable Endolysin Ply511.

The object is solved by the subject matter as defined in the claims.

The following figures illustrate the invention.

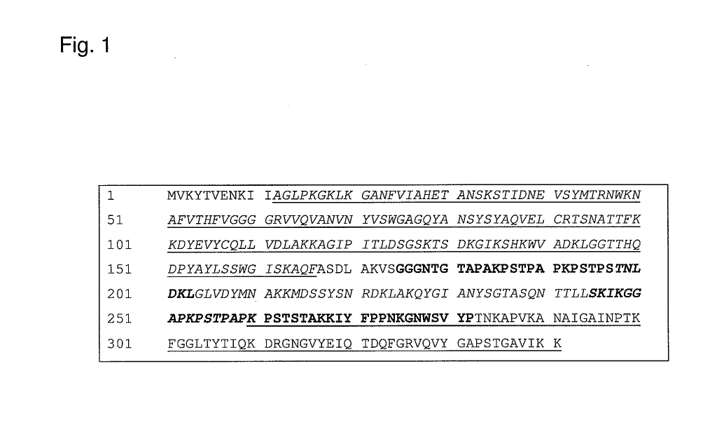

FIG. 1 shows the amino acid sequence of the Endolysin Ply511. The number of the first amino acid residue in each line is indicated on the left. The amino acid residues forming the EAD are italic and underlined. The amino acid residues forming the CBD1 are italic, the CBD2 are underlined. K260 is the last amino acid residue of CBD1 and the first of CBD2 at the same time. The domain linker sequences between EAD and CBD1 (amino acid residues 175 to 203) and between CBD1 and CBD2 (amino acid residues 245 to 282) are bold.

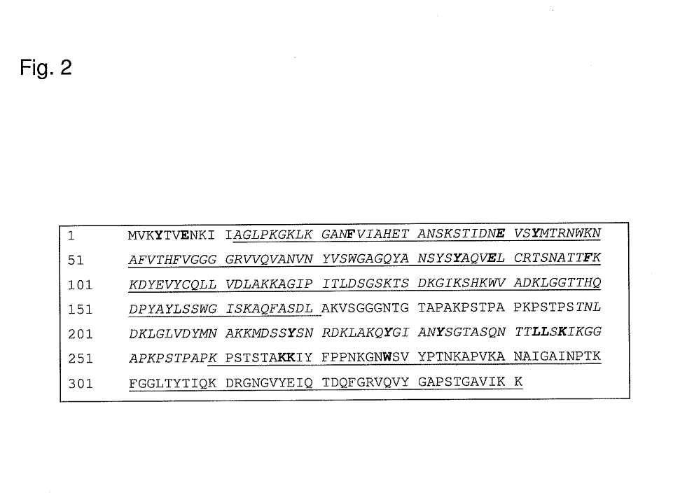

FIG. 2 shows the amino acid sequence of the Endolysin Ply511. The number of the first amino acid residue in each line is indicated on the left. The amino acid residues forming the EAD are italic and underlined. The amino acid residues forming the CBD1 are italic, the CBD2 are underlined. K260 is the last amino acid residue of CBD1 and the first of CBD2 at the same time. The amino acid residues in bold are cutting sites of proteases, determined by N-terminal sequencing of the emerging Endolysin fragment.

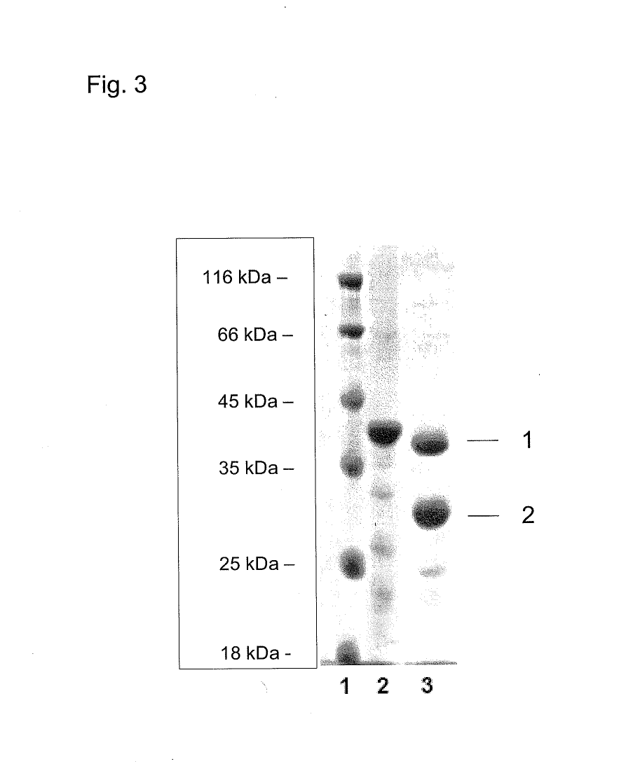

FIG. 3 shows the result of a polypeptide separation in a SDS-Polyacrylamide Gel after protease digestion during storage of Ply511. Lane 1 shows a molecular weight standard, lane 2 native full length Ply511, lane 3 Ply511 after storage. The band labeled with “1” is the full length Ply511, band “2” is a digested band. kDa means kilodalten.

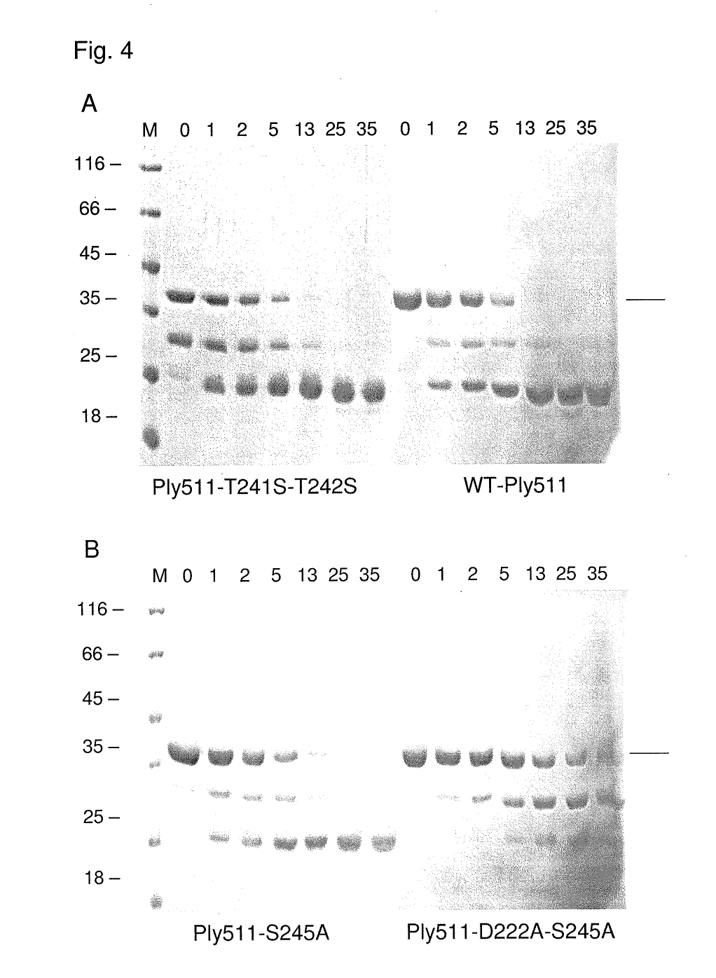

FIG. 4 shows the result of a polypeptide separation in a SDS-Polyacrylamide Gel after trypsin digestion, a comparison between Wt-Ply511 and mutants. FIG. 4A shows a comparison between the double mutant Ply511-T241S-T242S and Wt-Ply511. FIG. 4B shows the digestion of both mutants Ply511-S245A and Ply511-D222A-S245A. The numbers on the left are molecular weights in kilodalton. In the left lane (M) a molecular weight standard is loaded, respectively. The kinetic of the trypsin digestion is given in minutes above the gels above the corresponding lanes. The line on the right labels the position of the non-digested full length protein.

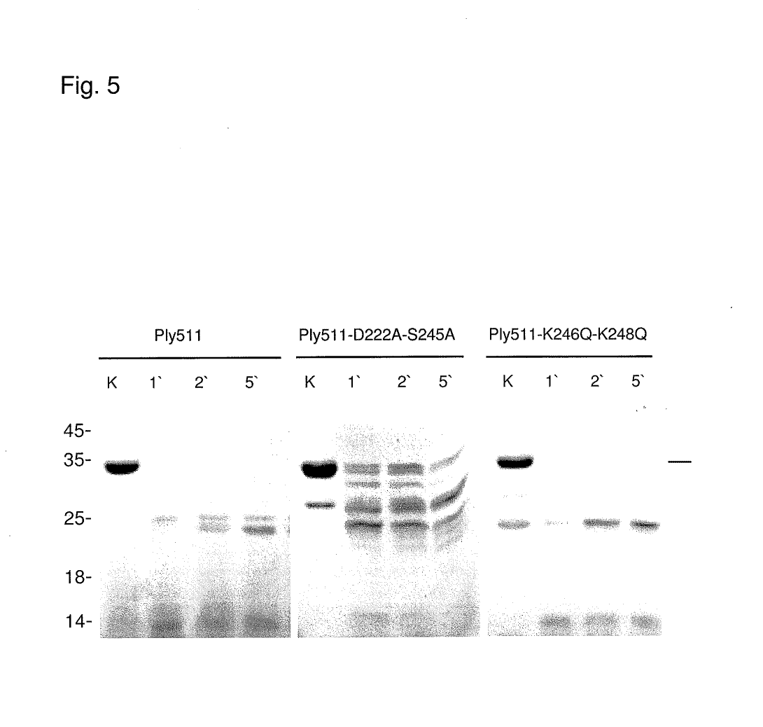

FIG. 5 shows the result of a polypeptide separation in a SDS-Polyacrylamide Gel after chymotrypsin digestion, a comparison between Wt-Ply511 and mutants. Wt-Ply511 as well as the mutants Ply511-D222A-S245A and Ply511-K246Q-K248Q were digested with chymotrypsin for one minute, two minutes or five minutes followed by loading on a SDS-gel. The control sample without adding chymotrypsin is labeled “K”. The numbers on the left are molecular weights in kilodalton. The line on the right labels the position of the full length protein.

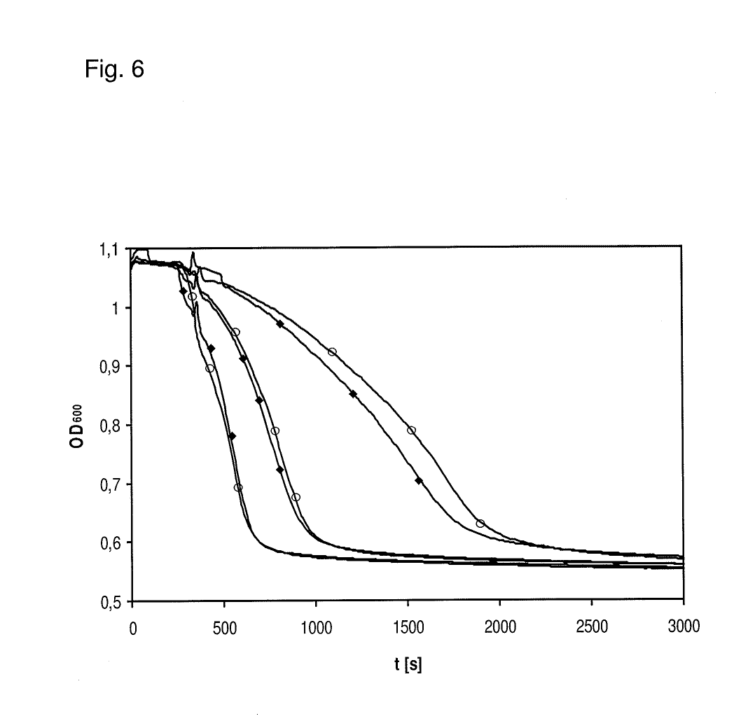

FIG. 6 shows a graphical diagram of the evaluation of a liquid lysis test, using Endolysin for the lysis of Listeria cells. Wt-Ply511 (∘) and the mutant Ply511-K246Q-K248Q (♦) in amounts of 0.1 μg, 0.3 μg or 0.7 μg (curves from left to right represent decreasing protein amounts, respectively) are added to heat inactivated cells of the Listeria strain 996 (Serotype 1/2b) and the decrease of the optical density at 600 nm (OD₆₀₀) as a function of time. t means time; [s] means seconds.

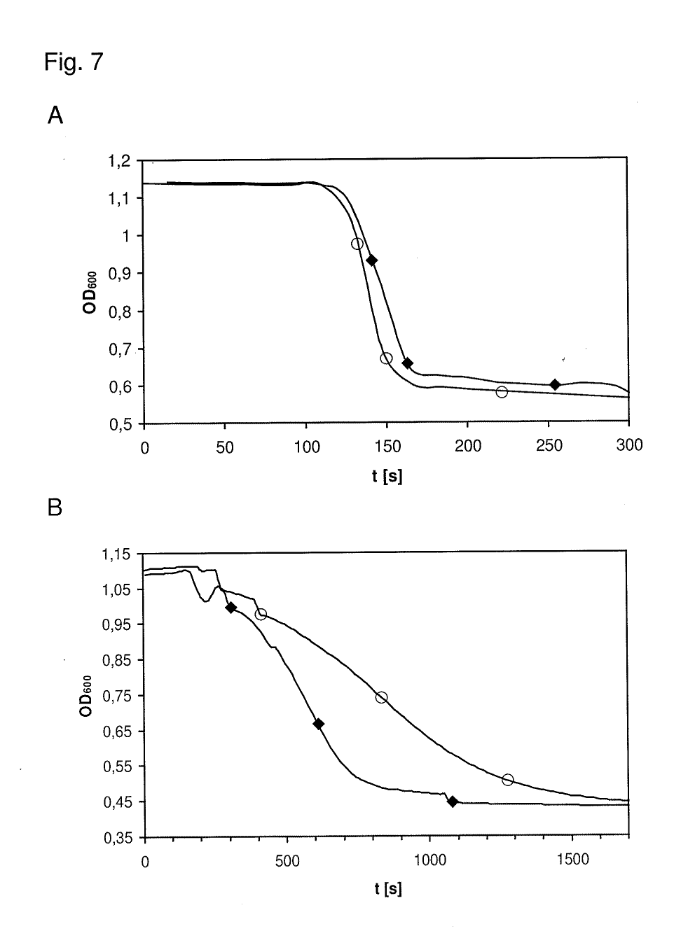

FIG. 7 shows a graphical diagram of the evaluation of a liquid lysis test, using Endolysin for the lysis of Listeria cells. Wt-Ply511 (∘) and a Ply511 mutant (♦) related to the present invention are added to heat inactivated cells of the Listeria strain 776 (Serotype 4b) and the decrease of the optical density was measured at 600 nm (OD₆₀₀) as a function of time. FIG. 7A Wt-Ply511 (∘) and mutants Ply511-G249A (♦) in a concentration of 10 μg/ml. FIG. 7B Wt-Ply511 (∘) and mutant Ply511-Δ195-262 (♦) in a concentration of 0.3 μg/ml. t means time; [s] means seconds.

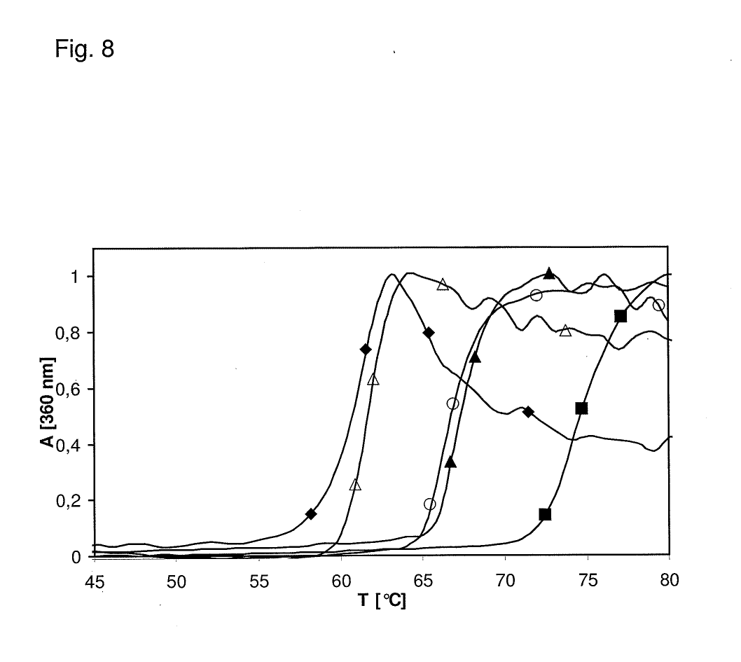

FIG. 8 shows a graphical diagram of the evaluation of a thermostability test of Wt-Ply511 and different mutants. Wt-Ply511 (∘) and the mutants Ply511-G249A (♦), Ply511-S245A (Δ), Ply511-D222A-S245A (▴) and Ply511-D222A (▪) were heated in the photometer and the increase of the protein aggregation (corresponds to an increase in absorption (A) at a wave length of 360 nm) was monitored as a function of temperature (T) in degree centigrade.

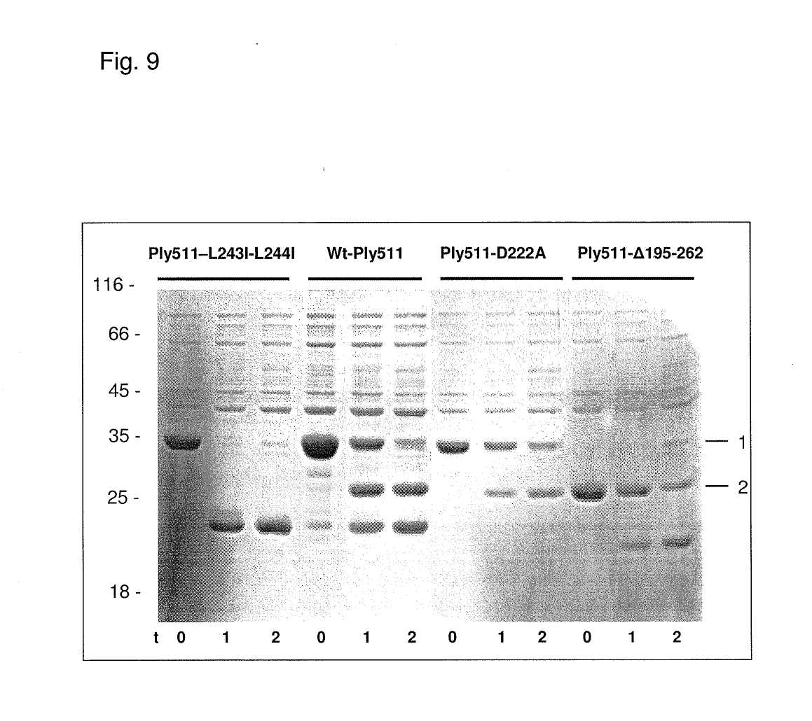

FIG. 9 shows the result of a polypeptide separation in a SDS-Polyacrylamide Gel after a protease digestion in an E. coli crude lysate. Wt-Ply511 and the mutants Ply511-L243I-L244I, Ply511-Δ195-262 as well as Ply511-D222A were expressed in E. coli and incubated for different time periods at 25° C. in the K coli lysate. The positions for the bands of the non-digested protein are indicated on the right (1: Ply511 full length protein, 2: truncated Ply511-Δ195-262). The numbers on the left are molecular weights in kilodalton. The numbers at the lower boarder are incubation times in days.

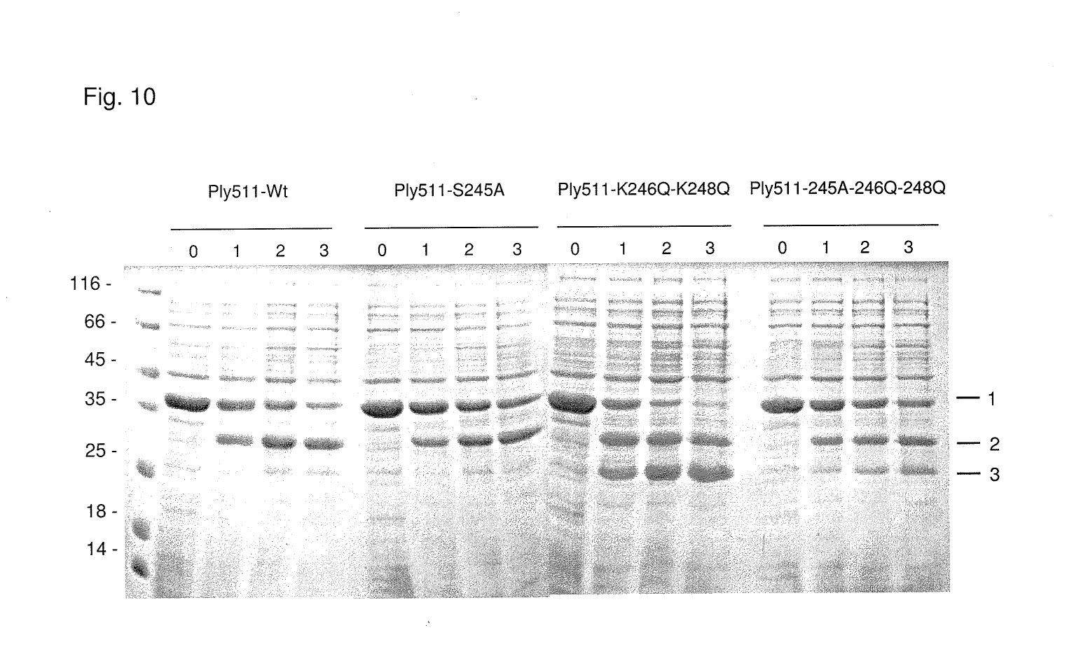

FIG. 10 shows the result of a polypeptide separation in a SDS-Polyacrylamide Gel after a protease digestion in an E. coli crude lysate. Wt-Ply511 and the mutants Ply511-S245A, Ply511-K246Q-K248Q as well as Ply511-S245A-K246Q-K248Q were expressed in E. coli and incubated for different time periods at 25° C. in the E. coli crude lysate. The positions of the bands for the non-digested protein (−1) as well as two prominent digestion fragments (−2, −3) are indicated on the right. The numbers on the left are molecular weights in kilodalton.

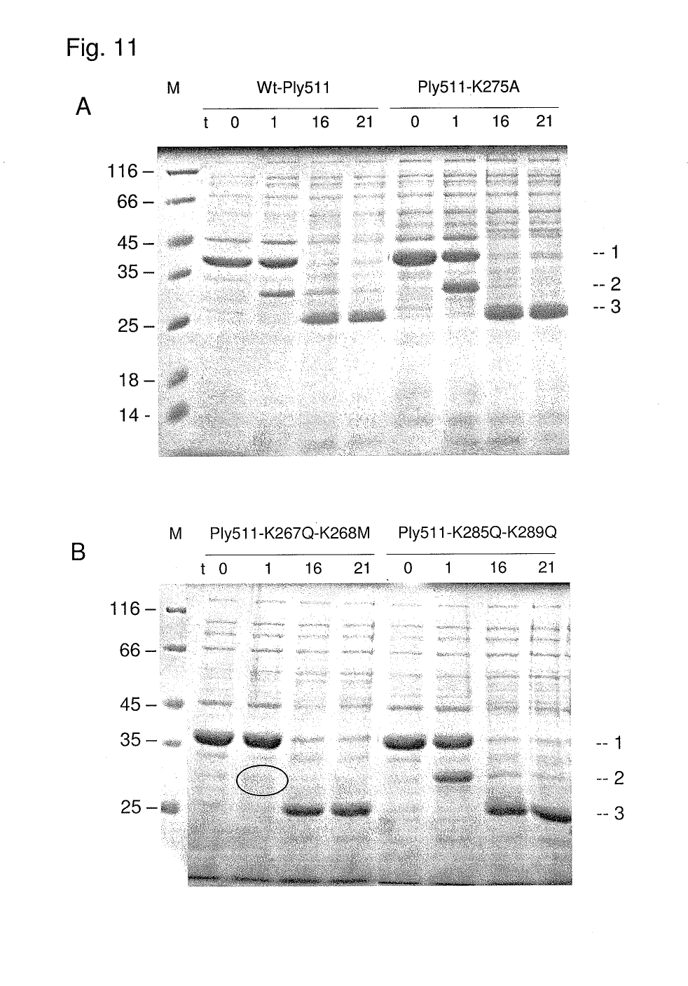

FIG. 11 shows the result of a polypeptide separation in a SDS-Polyacrylamide Gel after protease digestion in an E. coli crude lysate. Wt-Ply511 and the mutants Ply511-K275A (FIG. 11 A), Ply511-K267Q-K268Q as well as Ply511-K285Q-K289Q (FIG. 11B) were expressed in E. coli and incubated for different time periods at 25° C. in the E. coli crude lysate. The positions of the bands for the non-digested protein (−1) as well as two prominent digestion fragments (−2, −3) are indicated on the right. The numbers on the left are molecular weights in kilodalton. The oval labels the position where the mutant Ply511-K267Q-K268Q lacks the digestion intermediate of the size of approx. 28 to 30 kDa.

FIG. 12 shows the amino acid sequence of the Endolysin Ply511. The potential cutting sizes (R in P1 position) for the protease Clostripain are underlined. The both particularly sensitive cutting sites at the amino acid positions R62 and R221 determined experimentally are underlined and bold.

The term “protease” as used herein means an enzyme capable to hydrolytically cleave peptide bonds of proteins and/or peptides. The term comprises peptidases cleaving single amino acid residues from the amino- or carboxyl-terminus, as well as proteinases cleaving within a protein or polypeptide.

The term “wild type” or “Wt” as used herein means an amino acid sequence of the Endolysin Ply511 of the phage A511 as depicted in SEQ ID NO:1. The term also means the nucleotide sequence encoding the amino acid sequence according to SEQ ID NO:1. The nucleotide sequence isolated from the phage A511 encodes the Endolysin Ply511 and is depicted in SEQ ID NO:2. The term also includes the nucleotide sequence, which includes other codons as the one depicted in SEQ ID NO:2 for single amino acid residues, but encoding the same amino acid sequence due to the degenerated code.

The term “mutation” as used herein means an alteration of the initial amino acid sequence. Thereby single or more consecutive or by non-changed amino acid residues interrupted amino acid sequences may be deleted, inserted or added, or substituted. The term also includes a combination of the above mentioned single changes. The term also includes the N- or C-terminal fusion of a protein- or peptide-tag.

The term “modification” as used herein may be used as a synonym for “mutation”; however, the term additionally comprises chemical changes of the amino acid residues, e.g. biotinylation, acetylation, chemical changes of the amino-, SH- or carboxyl-groups.

The term “deletion” as used herein means the removal of 1, 2 or more amino acid residues from the respective initial sequence. In the following, the removed amino acid residues are indicated after the symbol “Δ”: e.g. “Δ195-262” means that the amino acid residues from position 195 inclusively to position 262 inclusively are removed from the initial sequence.

The term “insertion” or “addition” as used herein means the addition of 1, 2 or more amino acid residues to the respective initial sequence.

The term “substitution” as used herein means the exchange of an amino acid residue present at a certain position by another amino acid residue. In the following, substitutions are depicted as follows: After the changed amino acid residue in the one letter code, the position of the changed amino acid residue is depicted followed by the inserted new amino acid residue in the one letter code. Y4A means for example that the amino acid residue tyrosine at position 4 was changed to the amino acid residue alanine.

The term “domain” or “protein domain” as used herein means a subregion of an amino acid sequence exhibiting either a certain functional and/or structural feature. Due to amino acid sequence homologies, domains may frequently be predicted by computer programmes comparing amino acid sequences of free available databases with known domains; e.g. conserved domain database (CDD) at the NCBI (Marchler-Bauer et al., 2005, Nucleic Acids Res. 33, D192-6), Pfam (Finn et al., 2006, Nucleic Acids Research 34, D247-D251) or SMART (Schultz et al., 1998, Proc. Natl. Acad. Sci. USA 95, 5857-5864, Letunic et al., 2006, Nucleic Acids Res 34, D257-D260).

The term “domain linker” as used herein means an amino acid sequence having the function of linking between single protein domains. In general domain linkers do not or rarely form regular secondary structure elements such as α-helix or β-pleated sheet and could form different conformations in a respective structural context. The state of the art describes features of linker sequences as well as methods for their identification (George & Hering a, 2003, Protein Engineering, 15, 871-879, Bae et al., 2005, Bioinformatics, 21, 2264-2270).

The wild type Endolysin Ply511 exhibits a length of 341 amino acid residues. It has three functional domains each exhibiting homologies to other known Endolysins. The N-terminal amino acid residues at the positions 12 to 166 represent the enzymatic active domain (EAD) with the function of a N-acetylmuramoyl-L-alanine-amidase belonging to the group of the amidases 2. The cell binding domain (CBD) of Ply511 is split in two parts. A first CBD (CBD1) comprising the amino acid positions 198 to 260 exhibits similarities to the CBD of the Endolysin Ply118 of the Listeria phage A118. A further C-terminal positioned CBD (CBD2) comprising the amino acid positions 260 to 341 exhibits similarities to the CBD of the Endolysins from the bacillus phage Φ105. The single domains are linked with a domain linker. Domain linkers are located between the EAD and CBD1 in the region of the amino acid residues 175 to 203 and between the two CBDs in the region of the amino acid residues 245 to 282.

It turned out that during the recombinant expression of Wt-Ply511 in E. coli in addition to the full length protein a number of different fragments arise. This even applies to purified Ply511 if it is stored for a longer time. This loss of stability is associated with a loss of activity so that large amounts of protein have to be introduced to achieve a sufficient activity. To stabilize the Wt-Ply511 Endolysin, it was analyzed if there are certain regions within the Ply511 that are particularly sensitive towards proteases. The obtained fragments of Ply511 were separated by their size via SDS-Polyacrylamide Gel Electrophoresis followed by elution of the defined protein bands from the gels. The cutting sites of the proteases were determined by using N-terminal sequencing of the polypeptides of the respective bands. Additionally to the fragments occurring in the E. coli lysate or in the purified protein, where it is unknown which protease is responsible for the degradation, protease digestions with commercially available proteases (e.g. chymotrypsin, subtilisin, trypsin, pepsin, staphylococcus peptidase I, proteinase K) were also performed, where it is known after which amino acid residues they preferably cut. It turned out that the protease cutting sites were not uniformly distributed within the Endolysin, but certain regions where particularly sensitive. The proteins were cut frequently in the region of the N-terminus located upstream of the beginning of the EAD. Several cutting sites were also found within the EAD and CBD1 and within the linker between CBD1 and CBD2. Numerous protease cutting sites are present within the amino acid sequence LLSKIK comprising the amino acid positions 243 to 248 and located at the C-terminal end of the CBD1.

The present invention therefore relates to polypeptides exhibiting a changed amino acid sequence compared to the naturally occurring Endolysin Ply511 with the amino acid sequence according to SEQ ID NO: 1. The present invention further relates to the polypeptides according to the present invention, additionally comprising modifications. The present invention further relates to the nucleotide sequences encoding for the polypeptides according to the present invention. The polypeptides according to the present invention exhibit the lytic activity of the Wt-Ply511 Endolysin wherein the activity could be higher, equal or lower but not completely lost. The activity is measured with assays, known by a person skilled in the art, e.g. the plate lysis test or the liquid lysis test.

The alterations in the amino acid sequence may be deletions, insertions and additions, respectively, substitutions or combinations thereof.

Deletions introduced in the amino acid sequence of the naturally occurring Ply511 according to SEQ ID NO: 1 should preferably truncate the amino acid sequence such that protease cutting sites are removed without loss of the activity of the protein.

The deletions may affect one or more amino acid residues. If more amino acid residues are deleted, the deleted amino acid residues may be consecutive. Single, deleted amino acid residues or regions with more deleted amino acid residues may further be separated by one or more non-deleted amino acid residues. One or more deletions may therefore be introduced into the initial sequence of Ply511 according to SEQ ID NO: 1.

Deletions are preferably introduced into the region of the amino acid positions from 186 to 341 of the amino acid sequence according to SEQ ID NO: 1, especially into the region of the amino acid position 186 to 341, 195 to 255, 195 to 262, 238 to 341, 241 to 341, 267 to 341 and 270 to 341 of the amino acid sequence according to SEQ ID NO:1. Particularly preferred are deletions in the amino acid sequence according to SEQ ID NO:1, C-terminal of the position 237, particularly preferred C-terminal of the position 266, wherein the deleted region affects the indicated position to the end of the protein, thus to the amino acid position 341. Further preferred are deletions affecting only one part of the C-terminus, particularly deletions of the amino acid residues 195 to 262 and 195 to 255 according to the present amino acid sequence SEQ ID NO:1. These deletion polypeptides may be expressed completely soluble and show an increased activity compared to the Wt-Ply511 in the plate lysis test as well as in the liquid lysis test. Furthermore, the proteins were more stable towards protease degradation.

Surprisingly, it turned out that the N-terminal deletions within the region of the amino acid positions from 1 to approx. 11, particularly from 2 to 9, significantly decrease not only the solubility of the protein, but its activity is also completely lost.

Preferred polypeptides according to the present invention are summarized as examples in table 1.

TABLE 1

| | | Lysis of bacteria strain | | | |
|---|---|---|---|---|---|
| Mutation | Solubility | 776 | 996 | 1095 | 1147 |
| 6xH-Ply511- | + | ++ | ++ | ++ | ++ |
| Δ186-341 | | | | | |
| Ply511-Δ270-341 | + | ++ | +++ | ++ | +++ |
| Ply511-Δ267-341 | + | +++ | ++ | ++ | +++ |
| Ply511-Δ241-341 | + | −/+ | −/+ | −/+ | −/+ |
| Ply511-Δ238-341 | + | −/+ | −/+ | −/+ | −/+ |
| Ply511-Δ195-262 | + | ++++ | +++ | ++ | ++++ |
| Ply511-Δ195-255 | + | ++++ | +++ | ++ | ++++ |

776: Listeria monocytogenes Scott A (Serotype 4b)
996: Listeria monocytogenes (½b)
1095: Listeria monocytogenes (½a)
1147: Listeria innocua (6b)
Solubility: + like Wt-Ply511, − poorer than Wt-Ply511
−/+: Lysis activity barely detectable
+: Cell lysis significantly poorer than Wt
++: Cell lysis slightly poorer than Wt
+++: Cell lysis comparable to Wt
++++: Cell lysis better than Wt
6xH: N-terminal His-Tag with 6 histidine residues

The substitutions introduced into the amino acid sequence according to SEQ ID NO:1 of the naturally occurring Ply511 should preferably change the amino acid sequence such that protease cutting sites are removed without loss of the activity of the protein.

The substitution could affect one or more amino acid residues. If several amino acid residues are substituted, the substituted amino acid residues may be consecutive. Single substituted amino acid residues or regions with several substituted amino acid residues may further be separated from each other by one or several non-substituted amino acid residues. One or several substitutions may therefore be inserted into the initial sequence of Ply511 according to SEQ ID NO:1.

Preferred substitutions to remove protease cutting sites are Y4A and T5P. Further preferred substitutions at amino acid position 4 are G, T, S, C, I, V, E, Q, D, N, R and K. Further preferred substitutions are of any other amino acid residues for the E7 amino acid residue. Particularly preferred are the substitutions E7A and E7Q. Preferred are also Ply511-mutants with a combination of substitutions at the N-terminus and further substitutions, particularly Ply511-Y4A-E7Q, Ply511-T5P-E7A, Ply511-T5P-E7A-Δ195-262, Ply511-T5P-E7A-K246A, Ply511-Y4A-E7Q-K246A, Ply511-Y4A-E7Q-K246H and Ply511-Y4A-E7Q-Δ195-262. Further preferred are substitutions of all other amino acid residues except for R for the R92 and R221 amino acid residues, particularly the mutants with the substitutions Ply511—R92K-R221K and Ply511—R92A-R221A.

Preferred polypeptides according to the present invention are summarized as examples in table 2.

TABLE 2

Lysis of
bacteria strain

| Mutation | Solubility | 776 | 1147 |
|---|---|---|---|
| Ply511-Y4A | + | +++ | +++ |
| Ply511-T5P | + | +++ | +++ |
| Ply511-E7A | + | +++ | +++ |
| Ply511-E7Q | + | +++ | ++++ |
| Ply511-Y4A-E7Q | − | n.p. | ++ |
| Ply511-Y4A-E7Q-K246A | − | n.p. | +++ |
| Ply511-Y4A-E7Q-Δ195-262 | − | n.p. | ++ |
| Ply511-T5P-E7A | − (50 %.) | +++ | +++ |
| Ply511-T5P-E7A-Δ195-262 | − (50 %) | +++ | +++ |
| Ply511-T5P-E7A-K246A | + | ++ | ++ |
| Ply511-T5P-E7A-K246H | + | + | +++ |
| Ply511-R92K-R221K | + | ++ | +++ |
| Ply511-R92A-R221A | + | ++ | +++ |
| Ply511-MVKYTVENKI(1- | − (50 %) | − | − |
| 10)MASKKTNANK | | | |
| Ply511-MVKYTVENKI(1- | − (30 %) | − | − |
| 10)MASGGG | | | |

776: Listeria monocytogenes Scott A (Serotype 4b)
1147: Listeria innocua (6b)
Solubility: + like Wt-Ply511, − poorer than Wt-Ply511
n.p.: Experiment not performed
−: No Lysis activity detectable
+: Cell lysis significantly poorer than Wt
++: Cell lysis slightly poorer than Wt
+++: Cell lysis comparable to Wt
++++: Cell lysis better than Wt

It has been shown that the activity of the mutants Ply511-Y4A, Ply511-T5P, Ply511-E7A is equal to the activity of Wt-Ply511, whereas Ply511-E7Q exhibits an even higher activity. The multiple mutants Ply511-T5P-E7A-Δ195-262 and Ply511-T5P-E7A-K246A turn out to be of advantage whereas the other multiple mutants listed in table 2 still exhibit enzyme activity, but have a lower solubility compared to Wt-Ply511. If the first 10 amino acid residues of Ply511 (MVKYTVENKI) are exchanged for the amino acid residues MASKKTNANK (mutant Ply511-MVKYTVENKI(1-10)MASKKTNANK) or for the amino acid residues MASGGG (mutant Ply511-MVKYTVENKI(1-10)MASGGG), insoluble proteins with no activity arise, emphasizing again the importance of the N-terminus.

Further preferred are substitutions within the region of the EAD in the region of amino acid positions 12 to 166, particularly substitutions of acidic amino acid residues and aromatic amino acid residues. Preferred are substitutions at the amino acid positions 24, 43, 83, 92 and 99 selected from the group A, G, T, S, C, I, V, E, Q, D, N, R and K.

Preferred substitutions are summarized in table 3.

| | | Lysis of bacteria strain | | | |
|---|---|---|---|---|---|
| Mutation | Solubility | 776 | 996 | 1095 | 1147 |
| Ply511-F24I | + | ++++ | +++ | +++ | ++++ |
| Ply511-E40A | + | + | ++ | + | + |
| Ply511-E40Q | + | + | + | + | + |
| Ply511-Y43A | + | +++ | +++ | ++ | +++ |
| Ply511-Y43S | + | +++ | +++ | ++ | +++ |
| Ply511-E40Q-Y43S | + | − | + | − | + |
| Ply511-F24I-Δ195-262 | + | ++++ | ++++ | +++ | ++++ |
| Ply511-E40A-Δ195-262 | + | ++ | ++ | + | + |
| Ply511-E40Q-Δ195-262 | + | ++ | ++ | + | + |
| Ply511-Y43A-Δ195-262 | + | +++ | ++++ | ++ | +++ |
| Ply511-Y43S-Δ195-262 | + | +++ | +++ | ++ | +++ |
| Ply511-E40Q-Y43S- | + | − | + | + | + |
| Δ195-262 | | | | | |
| Ply511-Y83I | + | n.p | n.p | n.p | ++++ |
| Ply511-E89A | + | n.p | n.p | n.p | − |
| Ply511-E89Q | + | n.p | n.p | n.p | − |
| Ply511-R92K | + | ++ | n.p | n.p | +++ |
| Ply511-R92A | + | ++ | n.p | n.p | +++ |
| Ply511-F99A | − | n.p | n.p | n.p | +++ |

776: Listeria monocytogenes Scott A (Serotype 4b)
996: Listeria monocytogenes (½b)
1095: Listeria monocytogenes (½a)
1147: Listeria innocua (6b)
Solubility: + like Wt-Ply511, − poorer than Wt-Ply511
n.p: Experiment not performed
−: No Lysis activity
+: Cell lysis significantly poorer than Wt
++: Cell lysis slightly poorer than Wt
+++: Cell lysis comparable to Wt
++++: Cell lysis better than Wt

It has been shown that the substitutions at the positions 24, 43 and 83 have an advantageous effect, whereas substitutions at the positions 40 and particularly 89 have a negative effect on the activity. The mutants Ply511-Y43A, Ply511-Y43S are equal to the Wt-Ply511 concerning the activity, whereas the mutants Ply511-F241 and Ply511-Y831 are even more active than the Wt. This also applies to combinations of these substitutions, particularly in combination with the deletion Δ195-262. The mutant Ply511-F24I-Δ195-262 turned out to be particularly advantageous. After removing the protease cutting site of the mutant Ply511-F99A, the mutant remains active, but the solubility of the protein is reduced compared to the Wt. However, the mutation could be applied to increase the protease stability, if a slightly lower solubility is therefore accepted. The enzyme activity is negatively affected by the substitutions at the positions 40 and 89, particularly the mutants Ply51′-E40A, Ply511-E40Q, Ply51′-E89A, Ply511-E89Q barely exhibit activity or do not exhibit activity at all. This also applies to such combinations of mutations, where the single mutations have a positive effect on the function and stability of Ply511, e.g. Ply511-E40Q-Y43S, Ply511-E40A-Δ195-262, Ply511-E40Q-A195-262 and Ply511-E40Q-Y43 S-Δ195-262.

Further preferred are substitutions within the CBD1 in the region of the amino acid positions 198 to 260, particularly substitutions of the aromatic, basic and acidic amino acid residues at the positions 208, 218, 221, 222 and 228. Preferred substitutions at the amino acid positions 208, 218, 221, 222 and 228 are A, V, I, K, L and M. Further preferred is the substitution at position 222 from D to A.

Preferred substitutions are summarized in table 4.

TABLE 4

| Mutation | Solubility | Lysis of bacteria strain 1147 |
|---|---|---|
| Ply511-Y218V | + | +++ |
| Ply511-R221K | + | +++ |
| Ply511-R221A | + | +++ |
| Ply511-D222A | + | +++ |
| Ply511-Y228I | + | +++ |
| Ply511-Y233I | + | + |
| Ply511-Y233M | + | + |

1147: Listeria innocua (6b)
Solubility: + like Wt-Ply511, − poorer than Wt-Ply511
+: Cell lysis significantly poorer than Wt
+++: Cell lysis comparable to Wt

Whereas substitutions at the positions 218 and 228, particularly the mutants Ply511-Y218V and Ply511-Y2281, lead to a constant activity and the stability increases, the substitution at the position 233, particularly the mutants Ply511-Y2331 and Ply511-Y233M lead to a significant decrease of the activity and an even higher degradation in the E. coli lysate compared to the originally present amino acid residue Y. The substitution D222A is comparable to the Wt-Ply511 concerning the expression rate, solubility and activity, but surprisingly a significantly increased thermo stability of the mutant Ply511-D222A was shown compared to the wild type as well as an increased protease stability in the tryptic digest and in the E. coli lysate.

Further preferred are substitutions in the region of the amino acid positions from 243 to 248, exhibiting the amino acid sequence LLSKIK as well as the amino acid positions flanking this sequence N-terminal or C-terminal, particularly the region of the amino acid residues 240 to 249. Single as well as multiple mutations may thereby be introduced.

Preferred substitutions are summarized in table 5.

TABLE 5

| | | Lysis of bacteria strain | | | |
|---|---|---|---|---|---|
| Mutation | Solubility | 776 | 996 | 1095 | 1147 |
| Ply511-K246A | + | ++ | +++ | +++ | +++ |
| Ply511-K246H | − (70%) | ++ | +++ | +++ | +++ |
| Ply511-I247P | − (80%) | + | + | + | + |
| Ply511-L243I-L244I | + | n.p | n.p | n.p | ++ |
| Ply511-L243I-L244I-K246A | + | n.p | n.p | n.p | +++ |
| Ply511-L243I-L244I- | − | n.p | n.p | n.p | + |
| K246A-K248A | | | | | |
| Ply511-D222A-L243I-L244I- | + | n.p | n.p | n.p | + |
| K246A-K248A | | | | | |
| Ply511-K248A | + | n.p | n.p | n.p | +++ |
| Ply511-K246Q | + | n.p | n.p | n.p | +++ |
| Ply511-K248Q | + | n.p | n.p | n.p | +++ |
| Ply511-K246Q-K248Q | + | +++ | +++ | +++ | +++ |
| Ply511-L243I-L244I-K246Q | + | n.p | n.p | n.p | + |
| Ply511-L243I-L244I-K248Q | + | n.p | n.p | n.p | + |
| Ply511-L243I-L244I- | + | n.p | n.p | n.p | + |
| K246Q-K248Q | | | | | |
| Ply511-D222A-L243I-L244I- | + | n.p | n.p | n.p | + |
| K246Q-K248Q | | | | | |
| Ply511-D222A-K246Q-K248Q | + | n.p | n.p | n.p | +++ |
| Ply511-T241S-T242S | + | n.p | n.p | n.p | +++ |
| Ply511-D222A-T241S-T242S | + | n.p | n.p | n.p | +++ |
| Ply511-T241S-T242S- | + | n.p | n.p | n.p | ++++ |
| K246Q-K248Q | | | | | |
| Ply511-N240Q | + | n.p | n.p | n.p | ++ |
| Ply511-D222A-N240Q | + | n.p | n.p | n.p | ++ |
| Ply511-K246Q-K248Q-N240Q | + | n.p | n.p | n.p | ++ |
| Ply511-S245A | + | n.p | n.p | n.p | +++ |
| Ply511-D222A-S245A | + | n.p | n.p | n.p | +++ |
| Ply511-S245A-K246Q-K248Q | + | n.p | n.p | n.p | ++ |
| Ply511-G249A | + | n.p | n.p | n.p | ++ |
| Ply511-D222A-G249A | + | n.p | n.p | n.p | ++ |
| Ply511-K246Q-K248Q-G249A | + | n.p | n.p | n.p | + |
| Ply511-D222A-K246Q- | + | n.p | n.p | n.p | + |
| K248Q-G249A | | | | | |
| Ply511-N240A | + | n.p | n.p | n.p | + |
| Ply511-T241A | + | n.p | n.p | n.p | +++ |
| Ply511-T242A | + | n.p | n.p | n.p | +++ |
| Ply511-L243A | + | n.p | n.p | n.p | + |

776: Listeria monocytogenes Scott A (Serotype 4b)
996: Listeria monocytogenes (½b)
1095: Listeria monocytogenes (½a)
1147: Listeria innocua (6b)
Solubility: + like Wt-Ply511, − poorer than Wt-Ply511
n.p: Experiment not performed
−: No Lysis activity
+: Cell lysis significantly poorer than Wt
++: Cell lysis slightly poorer than Wt
+++: Cell lysis comparable to Wt
++++: Cell lysis better than Wt

A set of substitutions are suitable to improve the protease stability, but to maintain the enzyme activity of the Wt-Ply511. These are particularly the mutants Ply511-K246A, Ply511-K246H, Ply511-S245A, Ply511-T241A, Ply511-T242A and the double mutant Ply511-T241S-T242S. These substitutions also exhibit positive effects in combination with other mutations. The double mutation T241S-T242S in combination with the D222A and K246Q-K248Q also affects the activity in a slightly stabilizing and positive way. The mutation K246A is suitable to reduce the negative effect of the double-mutation L243′-L244I such that the activity of the triple mutant Ply511-L243I-L244I-K246A again reaches the level of the Wt-protein. The mutation S245A itself is already suitable for increasing the stability of the Ply511 in the E. coli crude lysate and for having a positive effect on the destabilizing double mutation K246Q-K248Q. The double mutant Ply511-D222A-S245A even exhibits significantly higher protease stability at a longer incubation time in the tryptic digest and in the E. coli lysate compared to the Wt-protein. However, the mutant Ply511-S245A is slightly destabilized in the thermo stability test compared to the wild type and just the combination of the mutations D222A and S245A lead to a protein with a stability comparable to the stability of the wild type in the thermo stability test. The enzyme activity and solubility of the protein is maintained by the mutants Ply511-K248A, Ply511-K246Q and Ply511-K248Q as well as the double mutant Ply511-K246Q-K248Q; however, the protease stability is decreased by these mutations.

Several substitutions within this sequence region also affect the enzyme activity and/or the protease stability in a negative way. These are particularly the mutations Ply511-I247P, Ply511-G249A, Ply511-N240Q, Ply511-N240A and Ply511-L243A as well as the double mutation Ply511-L243I-L244I. The mutant Ply511-G249A exhibits decreased thermo stability and protease stability during the purification; however, the enzyme activity is only slightly decreased compared to the wild type in the plate lysis test as well as the liquid lysis test. In all combinations with further mutations the double mutation Ply511-L243I-L244I also affects the protease stability and enzyme activity in a negative way. This also applies to combinations with the mutation N240Q as well as G249A.

Further preferred are substitutions within the CBD2 in the region of the amino acid position 260 to 341. Further preferred are substitutions in the mutant Ply511-Δ195-262 at the amino acid position 278. Further preferred substitutions are summarized in table 6.

TABLE 6

| | | Lysis of bacteria |
|---|---|---|
| | | strain |
| Mutation | Solubility | 1147 |
| Ply511_W278I | + | +++ |
| Ply511-K46A-W278I | + | +++ |
| Ply511-Δ195-262-W278I | + | ++ |
| Ply511-I247P-W278I | + | − |
| Ply511-K267Q-K268M | + | +++ |
| Ply511-K275A | + | +++ |
| Ply511-K285Q | + | +++ |
| Ply511-K289Q | + | +++ |
| Ply511-K285Q-K289Q | + | +++ |

1147: Listeria innocua (6b)
Solubility: + like Wt-Ply511, − poorer than Wt-Ply511
+++: Cell lysis comparable to Wt
++: Cell lysis poorer than Wt

The enzyme activity of the mutant Ply511-W2781 is sustained on the level of the Wt-protein; however, the protease sensitivity in the E. coli lysate is significantly poorer. This also applies to a combination of the mutation with further mutations. The mutations Ply511-K267Q-K268M, Ply511-K275A, Ply511-K285Q, Ply511-K289Q, of which particularly the double mutant Ply511-K267Q-K268M turned out to be suitable to increase the protease stability of the Ply511 Endolysins, at the same time an enzyme activity comparable to that of the Wt-protein is sustained. Particularly the mutations at the positions K267 and K268 for other amino acid residues except for R, particularly the mutant Ply511-K267Q-K268M turn out to be suitable to stabilize protease sensitive regions within CBD2.

Mutations increasing the protease stability of Ply511 are also suitable to increase the stability of fragments of the Endolysin Ply511 such as the EAD, the CBD1, the CBD2 or a combination of CBD1 and CBD2. The amino acid region comprised by the EAD is 1 to 166, the region comprised by the CBD1 is 198 to 260 and by CBD2 is 260 to 341 according to the sequence SEQ ID NO:1. The entire CBD therefore comprises the region from amino acid residue 166 on. The domains could further be truncated at the N- or C-terminus as long as they exhibit activity. For regions comprising the EAD, this activity is the lysis of Listeria cells (plate lysis test or liquid lysis test), for regions comprising just the CBD, this activity is not the lysis of the Listeria cells anymore, but only their binding, since CBD does not exhibit any amidase activity. All further above mentioned mutations, affecting the protease stability in a positive way and located within the described domain borders, are also suitable to stabilize the corresponding fragments of the Endolysin Ply511.

Modifications such as N- or C-terminal tags or chemical modifications of single amino acid residues may be added to facilitate the preparation of the proteins, e.g. His-Tag (Nieba et al., 1997, Anal. Biochem., 252, 217-228) or Strep-Tag (Voss & Skerra, 1997, Protein Eng., 10, 975-982) for easier purification, to improve its application, e.g. Strap-Tag, Avi-Tag (U.S. Pat. No. 5,723,584; U.S. Pat. No. 5,874,239), JS-Tag (WO 2008/077397) or chemical biotinylation for immobilisation on surfaces, exhibiting streptavidin or avidin or to increase the solubility or stability, e.g. PEGylation.

Preferably the invention further relates to the nucleic acid molecules encoding the described modified polypeptides according to the present invention. The present invention further relates to vectors, comprising the nucleic acid molecules according to the present invention as well as suitable host cells for the expression of the polypeptides according to the present invention.

The modified Ply511 Endolysins according to the present invention all exhibit a lysis activity, also exhibited by the naturally occurring Ply511. Furthermore, the above mentioned modifications cause positive effects, advantageously affecting a commercial application of the Endolysins. These positive effects may involve increased protease stability, thermo stability or stability towards chemical denaturants. The stabilization could further lead to higher expression rate, solubility or to a longer shelf life. The positive effect could further be an increased activity.

Increased protease stability is already important for the recombinant preparation of the protein. Due to the protease degradation which already begins with the preparation, the preparation of larger amounts of Ply511 is very difficult. Adding larger amounts of protease inhibitors would be expensive and involve a multitude of additive substances in the Endolysin preparation. Degraded protein could further be separated from the full length protein by sophisticated chromatography techniques; however, this would be difficult since a portion of the arising degradation fragments are just a few kilodalton smaller than the full length protein, such that the degradation fragments exhibit features similar to native protein concerning their purification. Increased protease stability is further important concerning the storage of the isolated Ply511. The protease stability is also desired concerning the use of Ply511 in food containing a multitude of proteases. Improved protease stability increases the duration of the efficiency of the added modified Ply511 according to the present invention.

Increased thermo stability is also advantageous. In food technology higher temperatures are often used, e.g. in cheese or yoghurt production. Ply511 Endolysin could here just be applied for the antimicrobic lysis of Listeria, if it is still active at appropriate temperatures. Increased thermo stability turns out to be also advantageous in the recombinant preparation of polypeptides according to the present invention. Proteins which are difficult to solubilize or instable have to be frequently expressed at low temperatures (e.g. 25° C. or 30° C.), such that the expression product is soluble. But an expression at higher temperatures (e.g. 37° C.) provides economic advantages since protein production is faster at these temperatures and higher cell densities could be achieved such that more protein could be produced.

Proteins exhibiting increased thermo stability, protease stability or also increased stability towards chemical denaturants are generally also stable to storage over a longer period of time. This turns out to be cost efficient for the manufacturer as well as the applying person, since larger amounts could be stored.

Good solubility of the protein is important to prepare the Endolysin in an efficient and cost-effective way. Insoluble protein is generally denatured and does not possess its native confirmation anymore and therefore its full activity. If the expression product is insoluble, refolding may be attempted to reobtain its native conformation and activity. However, this is technically sophisticated, expensive and inefficient concerning the yield of native protein such that preferably proteins with good solubility are expressed.

Higher activity is economically advantageous since fewer enzyme has to be applied.

The present invention further relates to the use of the proteins according to the present invention, as a human, veterinary and diagnostic substance, as antimicrobic substance in food or cosmetics or as disinfectant.

The present invention further relates to a pharmaceutical comprising a polypeptide according to the present invention. The present invention further relates to a pharmaceutical composition, comprising the polypeptide according to the present invention. A pharmaceutical composition according to the present invention could preferably comprise a pharmaceutically acceptable buffer, a pharmaceutical acceptable diluent or a pharmaceutically acceptable carrier substance. A pharmaceutical composition related to the present invention could further contain appropriate stabilisers, flavour additives or other appropriate reagents.

Another aspect of the present invention relates to the polypeptides according to the present invention for the use as a human, veterinary medical or diagnostic substance for therapy or prevention of diseases caused by Listeria or for diagnosis of Listeria contamination.

Diseases caused by Listeria comprise amongst others Listeriosis, gastroenteritis, meningitis, encephalitis, sepsis, local wound infection caused by smear infection and inflammation of the conjunctiva and cornea.

Another aspect of the present invention is the use of the polypeptide according to the present invention in a method for the treatment and/or prophylaxis of infections, particularly of infections caused by Listeria. This Listeria infection could particularly be an infection by L. monocytogenes, preferably by L. monocytogenes with the serotypes 1/2a, 1/2b, 1/2c, 3a, 3b, 3c, 4a, 4ab, 4b, 4c, 4d, 4e, 7, particularly by L. monocytogenes 1442 SV1/2 a, L. monocytogenes 1042 SV 4b, L. monocytogenes 1019 SV 4c and/or L. monocytogenes 1001 SV 1/2 c. This infection may further be a Listeria infection caused by L. innocua, preferably by L. innocua with the serotypes 3, 6a, 6b, 4ab, U/S, particularly by L. innocua 2011 SV 6a. The patient could be a human patient or an animal, preferably animals used in livestock breeding or in dairy farming, such as ruminants (e.g. cattle, cows, sheep and goats), pigs, horses, fowl, trapped wild birds, rabbits or predators. Preferably the polypeptides of the present invention are used in an appropriate amount at the location of the infection or at the location prophylactically treated against the infection.

Another preferred embodiment is the use of the polypeptide according to the present invention in a method for the treatment and/or prophylaxis of gastroenteritis, particularly of gastroenteritis caused by Listeria.

Another preferred embodiment is the use of a polypeptide according to the present invention in a method for the treatment and/or prophylaxis of Listeriosis, meningitis, encephalitis, sepsis as well as wound infection and inflammations of the conjunctiva and cornea caused by smear infection, particularly caused by Listeria.

Another preferred embodiment is the use of a polypeptide according to the present invention in a method for the treatment and/or prophylaxis of the above mentioned diseases during prenatal care.

A particularly preferred embodiment is the use of a polypeptide according to the present invention for the medical treatment, if the treated or prevented infection is caused by a resistant Listeria strain. A polypeptide of the present invention could further be used in methods for treatment of infections by the administration in combination with conventional anti bacterial active ingredients such as antibiotics, other enzymes such as e.g. Endolysins, etc.

The dosage and the mode of administration used in a method for the treatment and/or prophylaxes of the above mentioned diseases depends on the specific disease as well as the location of the infection, which should be treated. The mode of administration could in particular embodiments of the present invention be, e.g. oral, topical, parenteral, intravenous, rectal, or any other mode of administration. For the application of a polypeptide according to the present invention at the location of the infection (or the location, endangered to be infected), a polypeptide of the present invention may be formulated such that the polypeptide is protected from environmental influences such as proteases, from oxidation or from an immune response, etc.

A polypeptide of the present invention may therefore be present in a capsule, in a coated pill, in a pill, in a suppository, in an injectable solution or in any other medical appropriate galenic formulation. In several embodiments of the present invention, this galenic formulation may additionally contain suitable carriers, stabilisers, flavour additives, buffers, or other suitable reagents.

A polypeptide of the present invention could be administered for, e.g. a topical application as a lotion or a band-aid.

A suppository formulation could be used for the treatment of the intestine. Alternatively, an oral administration could be taken into consideration. In this case the polypeptide of the present invention has to be protected from environmental influences of the digestive system, until the polypeptide reaches the location of the infection. This could be achieved, e.g. by the use of bacteria as carriers, surviving the initial steps of gastric digestion and later releasing a polypeptide of the present invention in the environment of the intestine.

All medical applications base on the effect that the polypeptide of the present invention specifically and immediately lyse Listeria-bacteria after reaching them. This immediately influences the health of the treated patients by reduction of pathogenic bacteria and bacterial load and support of the immune system at the same time. For this purpose the same galenic formulations may be used, as for conventional medicaments for this application.

In another aspect, the polypeptides of the present invention are part of a cosmetic composition. A cosmetic composition according to the present invention may for example be used to inhibit or prevent irritations caused by a skin infection by Listeria-bacteria. A cosmetic composition according to the present invention preferably contains a sufficient amount of polypeptides according to the present invention to lyse already existing and/or recently settled Listeria-bacteria.

Another aspect of the present invention relates to the use of the polypeptides according to the present invention and/or host cells as antimicrobic substance in food such as, e.g. milk products, smoked fish, salted fish, frozen seafood, meat products, salads and ready-to-eat products (especially meat products and raw ready-to-eat products).

Another aspect of the present invention relates to the use of the polypeptides according to the present invention as antimicrobic substance in food-processing devices, in food-processing facilities, on surfaces exposed to food such as storage places, containers, or devices used for the storage or the processing of food and in all other situations where food may be contaminated potentially with Listeria-bacteria. In this context, the polypeptides according to the present invention may be used alone or in combination with other antimicrobic substances such as disinfectants, antibiotics or enzymes, e.g. such as other Endolysins.

The polypeptides according to the present invention may be applied to food products and/or different technical locations within food-processing facilities by a multitude of techniques, e.g. by mixing the polypeptides according to the present invention in the food-products, by spraying the polypeptides according to the present invention onto facility devices and/or directly applying the polypeptides according to the present invention onto facility devices.

Another aspect of the present invention is related to the use of the polypeptides according to the present invention in the diagnosis and the detection of Listeria contamination in medicine, food industry and food analytics, livestock breeding, analysis of drinking water or environmental analysis.

Listeria contaminations may be detected with the help of the polypeptides according to the present invention in different samples, e.g. in liquid solutions and mixtures of water and organic solvents, food, media, blood, blood products, plasma, serum, urine, stool samples, protein solutions, mixtures of water and ethanol as well as solutions containing non-liquid solid substances which should be analyzed or isolated, e.g. protein, DNA, RNA, sugar, salts, food, food-media-homogenates, pharmaceuticals, vaccines, organic and inorganic chemicals, e.g. NaCl, MgCl₂, purine and pyrimidine.

The following examples illustrate the invention and should not be understood as limited. If not specified, molecular biological standard methods where used as described by Sambrook et al., 1989, Molecular cloning: A Laboratory Manual 2. Auflage, Cold Spring Harbor Laboratory Press, Cold Spring Harbor, N.Y.

### EXAMPLE 1

### Test of Expression and Solubility

E. coli clones intended to be analyzed, containing the plasmids for Ply511 Endolysins were incubated under shaking in 1 ml LB-cultures at 30° C. until turbidity became visible. The cultures were induced with 1 mM IPTG except for the negative control. After 3-4 hours of incubation at 30° C., the cells were harvested in a table centrifuge (13,000 rpm for 10 mM at 4° C.). For expression tests the pellet was boiled (5 min at 95° C.) in 100 μl 1×SDS sample buffer and analyzed on SDS gels. For the solubility test, the pellet was resuspended in cell lysis buffer (25 mM Tris, 250 mM NaCl, pH 7.5) and lysed by sonication (20 s). After sedimentation of the insoluble proteins by centrifugation (13,000 rpm for 10 mM at 4° C.) sample buffer was added to aliquots of the supernatant (soluble protein fraction) and pellet (insoluble protein fraction) followed by boiling (5 mM at 95° C.). In both cases the samples were analysed by SDS Gel Electrophoresis followed by Coomassie staining of the gels.

### EXAMPLE 2

### Purification of Modified Endolysins Ply511 as Well as of Naturally Occurring Ply511

Ply 511 proteins were purified from cells of induced E. coli cultures (30° C., 1 mM IPTG). Cell pellets were lysed in loading buffer A1 (25 mM Tris, 250 mM NaCl, 1 mM MgCl₂, pH 8.0) with a micro fluidizer. After centrifugation, the supernatant was prepurified on a streamline direct HST-column (Cation exchange, GE healthcare). Therefore, 10 column volumes of buffer A1 and 10 column volumes of buffer A2 (25 mM borate, 250 mM NaCl, pH 9.0) were used for washing followed by buffer B1 (25 mM borate, 500 mM NaCl, pH 10.0) for elution. Phenylsepharose HP was used for a second purification step. The sample was loaded in buffer B4 (25 mM NaBorate, 1.1 M ammonium sulfate, pH 8.0), eluted with buffer AS (25 mM Na borate pH 8.0), with the Ply511 derivates being present in the flow-through. Salts were removed subsequently by dialysis against 40 mM Tris, 100 mM NaCl, pH 8.0 at 4° C., the buffer was changed twice within approx. 18 hours.

### EXAMPLE 3

### Analysis of Degradation Bands after Storage of Ply511

Purified Ply511 was incubated for two days at 4° C. in storage buffer (20 mM Tris, 500 mM NaCl, pH 8.0) and subsequently analysed on SDS gels in comparison to recently purified Ply511. It has been shown that during storage, a prominent degradation band of approx. 26 kilodalton and several smaller degradation bands arose, thus the protein was degraded by a protease and the protein is not stable during storage for a longer period of time. Protein preparations with protease degradation show a lower activity as the full length protein.

### EXAMPLE 4

### Identification of Protease Sensitive Regions within the Ply511 Sequence

To determine, which regions of the Endolysin Ply511 are exceptionally protease sensitive, protease digestion experiments with different commercially available proteases (e.g. chymotrypsin, trypsin, pepsin, subtilisin, staphylococcus peptidase I, proteinase K, thermolysin) were performed. Ply511 was incubated with protease at room temperature or 37° C., respectively, for different periods of time (minutes to several hours) in different buffers described by the manufacturers. The arising protease fragments were separated on SDS gels. The resulting protein bands were blotted on PVDF (Polyvinylidene fluoride) membranes, well discriminable bands were cut and N-terminally sequenced. Additionally to the commercially available proteases, the arising fragments of the E. coli lysate were also sequenced. Since fragments with similar size arose frequently, but not all fragments were sequenced and also further proteases with differing specificities exist, it has to be assumed that beside the mentioned positions of amino acids, also nearby amino acids are located within the protease sensitive regions.

### EXAMPLE 5

### Plate Lysis Test for Activity Analysis

For the preparation of lysis plates heat inactivated cells (20 min at 80° C.) of L. monocytogenes Scott A (Serotype 4b, strain no. 776), L. monocytogenes (Serotype 1/2b, strain no. 996 or Serotype 1/2a, strain no. 1095) or Listeria innocua (Serotype 6b, strain no. 1147) or further Listeria strains are added to LB-TopAgar such that a dense, turbid cell layer arises. If the lysis activity of transformed E. coli clones should be tested, LB-TopAgar contained IPTG and Ampicillin. To test the lysis activity either E. coli clones, transformed with plasmids for modified Ply511 variants, cell lysates of induced E. coli clones or purified protein solutions were subsequently dapped onto the plates (approx. 5 μl solution, inoculation loop of single colony of E. coli) followed by incubation overnight at 30° C. If the Ply511 Endolysins show lysis activity, a lysis area will become present at the sites where the protein was dapped onto the plates, the lysis area become visible as holes in the dense bacterial cell layer. The size of the lysis areas corresponds to the activity of the proteins. The activity is described in relation to the activity of the Wt-protein. All activity data from the shown tables were determined with help of the plate lysis test and the symbols (+++, ++, +, +/−) were determined according to the size of the lysis areas in comparison to the Wt Ply511.

### EXAMPLE 6

### Liquid Lysis Test for Analysis of the Activity

Per liquid lysis approach 1 ml of heat inactivated cells (20 min at 80° C.) from a bacterial culture, incubated up to an OD₆₀₀ of 1.0+/−0.1, wherein the bacterial culture is L. monocytogenes Scott A (Serotype 4b), L. monocytogenes (Serotype 1/2b or 1/2a) or L. innocua (Serotype 6b) or further Listeria strains. The bacterial cultures were introduced in PBST (20 mM sodium phosphate, 120 mM sodium chloride, 0.5% Tween, pH 8.0) and loaded into cuvettes. After addition of Endolysin (protein concentration between 0.1 μg/ml and 10 μg/ml) the decrease of the OD₆₀₀ was measured as a function of time at 30° C. The respective cell suspensions without addition of Endolysin served as control. The activity was calculated as decrease of the absorption at 600 nm per minute (ΔA/min) as a function of protein amount in μmol (ΔA μmol/min). The activities of the modified Endolysins were measured in comparison to the Wt-Ply511, respectively. In a comparative lysis test with Wt-Ply511 and Ply511-K246Q-K248Q with the Listeria strain 996 (Serotype 1/2b) 0.1 μg, 0.3 μg or 0.7 μg Endolysin was added, respectively. With increasing protein amount the lysis of Listeria became faster, but the Wt-Ply511 as well as the mutant Ply511-K246Q-K248Q showed approximately the same lysis activity at all three protein concentrations. In further liquid lysis tests comparative lysis data between Wt-Ply511 and mutants in comparison to the Listeria strain 776 (Serotype 4b) were determined. Wt-Ply511 and the mutant Ply511-G249A (10 μg/ml) also showed a very similar lysis activity, whereas the mutant Ply511-Δ195-262 showed an even faster lysis in comparison to the Wt-Ply511 (concentration 0.3 μg/ml).

### EXAMPLE 7

### Protease Digestion in the E. coli Lysate for Testing the Protease Stability

Induced 1 ml cultures of E. coli were harvested (13,000 rpm, 10 min, 4° C.) after 3-4 hours of incubation at 30° C. Subsequently the pellet was resuspended in cell lysis buffer (25 mM Tris, 250 mM NaCl, pH 7.5) and lysed by sonication (20 s). After sedimentation of the insoluble components and non-lysed cells via centrifugation (13,000 rpm, 10 min, 4° C.), the supernatant of the cell lysate was incubated at room temperature or 37° C. Samples were taken at the beginning of the incubation time (t=0), then at room temperature every 24 h (t=1, 2 or 3 days), samples incubated at 37° C. were taken within 24 hours (e.g. t=1, 16, 21 h). After boiling in SDS sample buffer (5 min, 95° C.) the samples were analyzed on SDS gels. Whereas the Wt-Ply511 showed a significant protein degradation at 25° C. by E. coli endogenous proteases and after two days almost no full length protein was present any more, the mutant Ply511-D222A and the Ply511-Δ195-262 showed a significantly delayed degradation such that after two days of incubation, full length protein was still present and the second degradation band with a smaller molecular weight did not appear at all within that time. However, the double mutant Ply511-L243I-L244I was significantly destabilized such that already after two days of incubation at 25° C. only protein with a molecular weight of the smaller degradation band (smaller 25 kDa molecular weight) was present. Another protease digestion in E. coli was incubated for up to 3 days at 25° C. Whereas the Wt-protein showed more and more of a decrease of the band of the full length protein (band 1) and the degradation band (band 2) showed an increase, the mutant Ply511-S245A showed that a significantly higher amount of the full length protein was still present. However, the double mutation K246Q-K248Q destabilizes the protein such that after 3 days basically no full length protein is present anymore, but a degradation band with smaller molecular weight (band 3) appears. The mutation S245A in combination with the double mutant K246Q-K248Q again has a stabilizing effect such that with the triple mutant Ply511-S235A-K246Q-K248Q full length protein was present until the end of the experiment and at the same time the degradation band 3 was populated to a lesser extent. It has been shown that within the CBD2 a trypsin sensitive cutting site was present leading to a degradation fragment with a size of approx. 28 to 30 kDa. It has been tried by introduction of different mutations to find and stabilize these protease cutting sites. Wt-Ply511 as well as the mutants Ply511-K275A, Ply511-K267Q-K268M and Ply511-K285Q-K289Q were incubated in the E. coli lysate at 37° C. for 1, 16 or 21 hours. All of them showed protease stabilities which were at least equal to the Wt-Ply511. However, it has been shown that together with the potential trypsin cutting site in the mutant Ply511-K267Q-K268M a universal protease cutting site was also removed, since the degradation intermediate of approx. 28 to 30 kDa was not present within this mutant.

### EXAMPLE 8

### Trypsin Digestion For Testing The Protease Stability

Wt-Ply511 (SEQ ID NO:1) and the tested mutants (Ply511-T241S-T242S, Ply511-S245A and Ply511-D222A-S245A) were purified as described in example 2. They were dialysed twice approx. 18 hours in total against 25 mM sodium phosphate, 100 mM NaCl, pH 8.0 before the protease digestion. The dialysis buffer was also used for the tryptic digest. 30 μg of Endolysin was introduced in a sample volume of 150 μl. 2.5 μl of a trypsin stock solution (1 mg/ml in 25 mM sodium phosphate, 100 mM NaCl, pH 8.0) were introduced into the digestion step and digested for 1 min, 2 min, 5 min, 13 min, 25 min and 35 mM at room temperature. Respective samples were taken at the mentioned time points, sample buffer was added and subsequently all samples were analyzed on a 12% SDS gel. A sample not incubated with trypsin served as control. Whereas the double mutant Ply511-T241S-T242S is degraded with a similar kinetic to the Wt-protein, both mutants Ply511-S245A and particularly Ply511-D222A-S245A are stabilized against protease degradation. The kinetic of the degradation is significantly slower. Mainly two defined degradation bands with a molecular weight of approx. 29 kDa and 26 kDa arise, respectively. It should be noted that the protease stability towards trypsin of the described mutants increased, although no amino acids were exchanged representing direct cutting sites for trypsin (lysine and arginine). That means that the described mutations stabilize the protein against proteases in general and not only in the sense that certain cutting sites for certain sequentially determined proteases were removed.

### EXAMPLE 9

### Chymotrypsin Digestion for Testing the Protease Stability

Wt-Ply511 and the mutants Ply511-D222A-S245A and Ply511-K246Q-K248Q were purified as described in example 2. They were dialysed twice approx. 18 hours in total against 25 mM sodium phosphate, 100 mM NaCl, pH 8.0 before the protease digestion. The dialysis buffer was also used for the digestion with chymotrypsin. 24 μg Ply511 were incubated with 3 μg chymotrypsin for 1 mM, 2 min or 5 min in a sample volume of 150 μl at room temperature, added to SDS sample buffer at the mentioned time points and subsequently analyzed on a 12% SDS gel. A sample which was not incubated with chymotrypsin served as control. Whereas the double mutant Ply511-K246Q-K248Q was degraded slightly faster than the Wt-protein the mutant Ply511-D222A-S245A is significantly stabilized against the protease degradation.

### EXAMPLE 10

### Thermo Stability Test for Protein Aggregation

For the thermo stability test 100 μg of the respective protein was introduced in 25 mM Na-phosphate, 100 mM NaCl, ph 8.0 and loaded into a stirable quartz cuvette (volume 1 ml). The increase of the optic density (light diffusion by aggregation of the protein) during the heating from 20 to 90° C. (beating rate 1° C./min) were measured in the photometer at a wave length of 360 nm. In an exemplified experiment Wt-Ply511 and the mutants Ply511-G249A, Ply511-S245A, Ply511-D222A-S245A and Ply511-D222A were heated in the photometer and the increase of the protein aggregation was measured as a function of temperature. It has been shown that Wt-Ply511 aggregates at approx. 65° C. whereas the destabilizing G249A and S245A in the mutants Ply511-G249A and Ply511-S245A leads to aggregation already at 60° C. In contrast, the mutant Ply511-D222A is significantly more thermo stable and aggregates not until approx. 75° C. If the mutations D222A and S245A are combined, the double mutant Ply511-D222A-S245A shows slightly increased thermo stability compared to the Wt such that the effects of the single mutations behave more or less additive.

### EXAMPLE 11

### Thermo Stability Test for Protein Activity

To test if potentially higher thermo stability affects the protein activity, different Ply511 variants (protein concentration 0.3 mg/ml) were incubated each for 20 min at increased temperature in buffer (40 mM tris, 100 mM NaCl, pH 8.0) and subsequently the rest activity was determined in the liquid lysis test (see example 6). Thereby the activity is related to the decrease of the absorption at 600 nm per min (ΔA/min) in the initial phase of the lysis curve. Wt-Ply511 and the mutant Ply511-G249A (concentration 3 μg/ml each) were incubated for 20 min in PBST at 50° C. whereas the controls were stored at 4° C. After that period of time the rest activity was measured in the liquid lysis test at room temperature. It has been shown that Wt-Ply511 kept 98% of its activity under these conditions whereas the mutant Ply511-G249A only showed 15% rest activity.

### EXAMPLE 12

### Stability Against Chemical Denaturants

Proteins in their native form show characteristic fluorescence emission spectra. During denaturation in chemical denaturants such as guanidinium chloride (GdmCl) or urea, the position of the emission maximum as well as the intensity of the fluorescence signal changes. The protein fluorescence is measured as a function of addition of denaturant at the wave length, which leads to the biggest change in signal between the native and the denatured protein, to get information about the stability of a protein. The protein is more stable if the mid point of the denaturation transition of a protein is higher (in M denaturant). The stability of the modified Ply511 Endolysins is compared with the stability of the Wt-protein, respectively. GdmCl-stock solutions are prepared in water between 0 and 8 M in steps of 0.5 M and the concentration of the denaturant is subsequently controlled in a refractometer. A protein stock solution is prepared at 100 μg/ml in four times concentrated PBS buffer (PBS: 20 mM sodium phosphate, 120 mM sodium chloride, pH 8.0). The GdmCl-stock solution as well as the PBS buffer is sterile filtered. Every 0.75 ml of the protein stock solution is mixed with 2.25 ml of the different GdmCl-stock solutions and the samples are incubated at 25° C. For measurement of the fluorescence 0.75 ml are taken from the respective samples followed by measuring the fluorescence signal. Thereby blank values for the buffer with the corresponding GdmCl concentration without adding protein are subtracted from each measuring point. To control if a steady state for the protein denaturation is already set, the measurement is repeated approx. after 7 days and repeated again later if necessary. The corrected fluorescence values are subsequently blotted against the concentration of the denaturant for detelmination of the mid point of denaturation.

### EXAMPLE 13

### Cell Binding Test for Non-Enzymatic Active Variants of Ply511, Particularly Fragments Containing CBDs

Ply511-CBD fragments are fused with N- or C-terminal tags such as his tags or strep tags and heterologously expressed in E. coli. For fluorescence detection of the bound CBDs a GFP marker could be fused in between the tags and the Ply511-CBD sequence. The proteins are purified with the help of the tag via affinity chromatography according to the manufacturers protocol. 50 μl of a preculture of L. monocytogenes Scott A (Serotype 4b), L. monocytogenes (Serotype 1/2b or 1/2a) or Listeria innocua (Serotype 6b) or further Listeria strains are mixed with approx. 2 μg of the purified proteins and incubated for 10 min at room temperature. After addition of 1 ml PBST (10 mM sodium phosphate, 150 mM NaCl, 0.05% Tween 20, pH 8.0) the cells are centrifuged, washed twice in 0.5 ml and resuspended in 50 ml PBST. Binding of Ply511-CBDs to Listeria-cells is controlled under the fluorescence microscope. For Ply511-CBD fusions with Strep-tags it is possible to use magnetic beads coated with streptavidin or avidin. In this case Strep-tag-Ply511-CBDs which are bound to bacteria are incubated with appropriate magnetic beads. Complexes of Listeria cells and Ply511-CBD are subsequently separated from the sample with the help of a magnetic separator. The detection of Listeria is then performed with conventional methods (e.g. PCR, microbic detection techniques).

### EXAMPLE 14

### Identification of Clostripain Cutting Sites in Listeria Endolysins

Potential cutting sites for Clostripain are frequently present in Endolysins in large quantities. Since a substitution of all potential cutting sites could influence the activity of the Endolysins in a negative way, it may be useful to determine the cutting sites which are accessible to the protease and only to modify these. The Listeria Endolysin Ply511 contains six potential cutting sites for Clostripain. A digest of Ply511 with Clostripain was performed to determine the Clostripain sensitive regions of the Endolysin. Ply511 (0.1 mg/ml) was digested for 3 hours and overnight, respectively, at room temperature with 5 units Clostripain (definition of units according to the manufacturer, Sigma) in 60 μl of sample volume with the following composition: 25 mM sodium phosphate, 1 mM calcium acetate, 2.5 mM DTT, pH 7.6. The arising protein fragments were separated by SDS gel electrophoresis (gradient gels 10-20% acrylamide). 3 bands arose (molecular weights approx. 25 kDa, approx. 14 kDa, approx. 10 kDa), were blotted on PVDF membranes, cut out subsequently and sequenced via N-terminal Edman degradation.

For the fragments the following N-terminal sequences emerged:
1. (M)V K Y T V E N K; the N-terminal methionine was partially dissociated
2. DKLAK
3. TSNATTF

This result shows that out of the 6 potential Clostripain cutting sites (R46, R62, R92, R221, R312, R326) two were recognized by the protease, namely R92 and R221. Variants of Ply511 stabilized according to the present invention possess exchanges of R to other amino acid residues at these positions, particularly R62K or R62A as well as R221K or R221A.

1. A polypeptide having the biological activity to lyse Listeria, wherein the polypeptide comprises an altered amino acid sequence having at least one amino acid position differing from the amino acid sequence according to SEQ ID NO:1.

2. The polypeptide according to claim 1, wherein the altered amino acid sequence is a deletion, addition and/or substitution.

3. The polypeptide according to claim 1, wherein the altered amino acid sequence is present at directly consecutive amino acid positions or at amino acid positions separated by one or more unchanged amino acid residues.

4. The polypeptide according to claim 1, comprising deletions in the region of the amino acid positions from 186 to 341 of the amino acid sequence according to SEQ ID NO:1.

5. The polypeptide according to claim 4, comprising deletions of the amino acid positions 186 to 341, 195 to 255, 195 to 262, 238 to 341, 241 to 341, 267 to 341 and 270 to 341 of the amino acid sequence according to SEQ ID NO:1.

6. The polypeptide according to claim 1, comprising one or more substitutions at the amino acid positions 4, 5, 7, 24, 43, 46, 83, 92, 99, 208, 218, 221, 222, 228, 246, 249, 267, 268, 275, 278, 285 or 289, or in the region of the amino acid positions from 12 to 166, 198 to 260, 240 to 249, 260 to 341.

7. The polypeptide according to claim 6, wherein the substitution at amino acid positions 4, 24, 43, 83, 92 and 99 is A, G, T, S, C, I, V, E, Q, D, N, R or K, at amino acid position 249 the substitution is A and at amino acid positions 208, 218, 221 and 228 the substitution is A, V, I, K, L or M.

8. The polypeptide according to claim 6, wherein the substitution at amino acid position 4 is A, at amino acid position 5 is P, at amino acid position 7 is A or Q, at amino acid position 24 is I, at amino acid position 43 is A or S, at amino acid position 83 is I, at amino acid position 92 is A or K, at amino acid position 99 is A, at amino acid position 221 is A or K, at amino acid position 222 is A, at amino acid position 246 is A or H, at amino acid position 245 is A, at amino acid position 241 is A, at amino acid position 242 is A, at amino acid position 248 is A or Q, at amino acid position 246 is Q, at amino acid position 275 is A, at amino acid position 278 is I, at amino acid position 285 is Q, at amino acid position 289 is Q, at amino acid position 267 and 268 is not R.

9. The polypeptide according to claim 6, wherein the polypeptide comprises substitutions at the amino acid positions Y4A and E7Q, or T5P and E7A, or TSP, E7A and K246A, or Y4A, E7Q and K246A, or Y4A, E7Q and K246H, or D222A and S245A, or T241S and T242S, or T241S, T242S and D222A, or T241S, T242S, K246Q and K248Q, or L243I, L244I and K246A, or S245A, K246Q and K248Q, or K246Q and K248Q, or K267Q and K268M, or K46A and W278I, or R92A and R221A or R92K and R221K.

10. The polypeptide according to claim 1, wherein the polypeptide comprises deletions and substitutions at the amino acid positions TSP, E7A and Δ195-262, or Y4A, E7Q and Δ195-262, or F24I and Δ195-262, or W278I and Δ195-262.

11. A nucleic acid molecule, wherein the nucleic acid molecule comprises a nucleotide sequence encoding a polypeptide according to claim 1.

12. The nucleic acid molecule according to claim 11, wherein said nucleic acid is located in a vector.

13. The nucleic acid molecule according to claim 11, wherein said nucleic acid is located in a host cell.

14. The polypeptide of claim 1, formulated as a human, veterinary medical or diagnostic substance, as antimicrobic substance in foods or in cosmetics, as disinfectant or as an environmental agent.

15. The polypeptide according to claim 14, wherein the foods are milk products, smoked fish, salted fish, frozen seafood, meat products, salads or ready-to-eat products.

16. The polypeptide of claim 1, formulated as a human, veterinary medical or diagnostic substance in the therapy and/or prevention of diseases caused by Listeria or for the diagnosis of Listeria contaminations.

17. The polypeptide according to claim 16, wherein the diseases caused by Listeria comprise Listeriosis, gastroenteritis, meningitis, encephalitis, sepsis; local wound infections caused by smear infections and inflammations of the conjunctiva and cornea.

18. The polypeptide according to claim 16, wherein said disease or contamination occurs during pregnancy.

19. A method of using the polypeptide according to claim 1 for detection of Listeria contaminations in medicine, in food industry and analytics, in live stock breeding, in drinking water or in environmental analytics comprising contacting the polypeptide with the medicine, food, analyte, live stock sample, drinking water or environmental sample with the polypeptide.

20. A method of using the polypeptide according to claim 1 as antimicrobic substance in foods or in cosmetics, as disinfectant or in the environmental field comprising contacting the food, cosmetic or environment with the polypeptide.

21. A method of using the polypeptide according to claim 1 as antimicrobic substance in food processing devices, in food processing facilities, on surfaces exposed to foods, and in facilities used for the storage or the processing of foods comprising contacting the food processing device, surface or facility with the polypeptide.

22. The method according to claim 21, further comprising contacting the food processing device, surface or facility with other disinfectants, antibiotics and/or enzymes.
